(19) United States
(12) Patent Application Publication
(10) Pub. No.: US 2012/0177592 A1
(43) Pub. Date: Jul. 12, 2012

(54) DEGRADABLE THERMORESPONSIVE POLY(ETHYLENE GLYCOL) ANALOGUE MATERIALS

(75) Inventors: Maciej Radosz, Laramie, WY (US); Youqing Shen, Laramie, WY (US)

(73) Assignee: UNIVERSITY OF WYOMING, Laramie, WY (US)

(21) Appl. No.: 12/601,914

(22) PCT Filed: May 30, 2008
(86) PCT No.: PCT/US08/65287

§ 371 Date: Mar. 27, 2012

Related U.S. Application Data
(60) Provisional application No. 60/932,203, filed on May 30, 2007.

(51) Int. Cl. A61K 47/48; C08G 69/00; C08G 63/78; C08G 63/52

(52) U.S. Cl. 424/78.17; 528/294; 528/290; 528/296; 528/288; 525/451; 525/418

(57) ABSTRACT

A method of developing degradable linear poly(ethylene glycol) PEG (DPEG) with multiple functioning capacities, which can be used as drug carriers for cancer cell delivery. A DPEG may be effective in targeting cancerous tumors through an enhanced permeation and retention effect (EPR). The DPEG will then degrade in the acidic extracellular fluid of solid tumors leading to fast cellular internalizations, finally degrading in the lysosome for efficient renal clearance. These may be used in conjunction with drugs and/or targeting groups. Furthermore, DPEGs are thermoresponsive, on an as needed basis, making them useful for in vivo application.

This application claims priority to U.S. Patent Application Ser. No. 60/932,203 filed May 30, 2007.

### BACKGROUND OF THE INVENTION

Over the past decades, water-soluble polymers¹⁻⁶ have been found to carry drugs preferentially to cancerous tissues, resulting in drug concentrations orders of magnitude higher than that in healthy tissues and thus significantly enhanced therapeutic efficacy while greatly reduced drug side effects.^{7, 8} The cancer-targeted drug delivery results from the enhanced permeation and retention (EPR) effect in cancerous tissues due to the leaky blood capillaries and an impaired lymphatic drainage.

Drugs bound to water-soluble polymers, namely, polymer drugs or polymer-drug conjugates,^{1-6, 9} exhibit much longer circulation times in the bloodstream for passive accumulation in cancerous tissues, lower toxicity to healthy tissues, wider dose windows,¹⁰ and much higher antitumor activity, and they can bypass cancer cells' membrane-associated multidrug resistance.¹¹⁻¹⁴ Various water-soluble polymers have been explored as drug carriers, including polyglutamates, albumin, poly[N-(2-hydroxypropyl) methacrylamide] (PHPMA), polyacetals, and dendrimers. Of the various available polymers for polymer-drug conjugates, PEG is most widely used due to its low toxicity and low interaction with proteins and cells. Many PEG-anticancer drug conjugates such as PEG-camptothecin.^{15, 16} PEG-doxorubicin,¹⁷⁻¹⁹ PEG-paclitaxel,²⁰ and PEG-methotrexate have been developed and some of them are in clinical trials.²¹

In the PEG-drug conjugates, the PEG molecular weight plays a major role in cancer targeting and cellular uptake. Passive accumulation of the conjugates in cancerous tissues via the EPR effect requires the PEG carrier to have long circulation time, and therefore, slow renal clearance.¹⁻⁶ Yamaoka reported that the renal clearance rate of PEG decreased with increasing its molecular weight, with the most dramatic decrease at 30,000.²² Thus, higher molecular weight PEG has a longer plasma residence time and consequently a greater tumor targeting.¹⁹ However, the in vitro cytotoxicity of the conjugates decreases with increasing the PEG molecular weight due to the decreased cellular uptake rate.^{6, 19, 21} Furthermore, nondegradable PEG with the molecular weight higher than its renal threshold may be retained in the body and may cause serious kidney damage and lysosomal storage disease syndrome upon repeated application.²³ In addition, linear PEG only has one or two terminal groups available for drug and targeting group conjugations.

Therefore, one objective of this invention is to develop a degradable linear PEG (DPEG) with multiple functional groups as drug carriers. Such a DPEG can be made to have high molecular weight for effective tumor targeting by the EPR effect, but degrade into shorter polymer chains in the acidic extracellular fluid of solid tumors (pH <7^{24, 25}) for fast cellular internalization, and further degrade in lysosomes (pH 4-5²⁶) for efficient renal clearance. The multifunctional groups in the DPEG can be used for conjugation of drugs and targeting groups, such as folic acid targeting groups. These DPEGs are also targeted to be used for pegylation.

Another objective of this invention is to make such DPEGs thermoresponsive, if needed, that is to make them precipitate from an aqueous solution upon heating. Synthetic thermoresponsive polymers are mainly poly(N-alkyl acrylamide)s, poly(vinyl ether)s, poly(N-vinylcaprolactam), polyethylene oxide-polypropylene oxide-polyethylene oxide (PEO-PPO-PEO) block copolymers, and poly(ethylene glycol) (PEG) brushes.²⁷⁻³¹ These polymers, however, are nondegradable and may not be used in vivo. Thermoresponsive DPEGs will be useful for in vivo applications

### SUMMARY OF THE INVENTION

A new class of poly(ethylene glycol) (PEG)-derived materials, degradable PEG analogues (DPEGs) are synthesized by condensation polymerization by either Michael Addition of PEG-di(meth)acrylates or di(meth)acrylamides with dithiols or PEG-diols or PEG-diamines with dianhydrides. DPEGs can be made to be fast degradable through hydrolysis, carry multiple functional groups such as thiol, (meth)acrylates, hydroxy and carboxylic acid groups. DPEGs can have lower critical solubility temperatures (LCSTs) tunable from 0 to 50° C. These DPEGs are useful as multifunctional water-soluble drug delivery carriers, for pegylation of biomolecules, biopolymers and colloidal particles. DPEGs can be used to develop a new class of thermoresponsive drug carriers. Crosslinked DPEGs are thermoresponsive hydrogels.

### BRIEF DESCRIPTION OF THE FIGURES

FIG. 1 is an illustration of in vitro hydrolysis of PEGDA258-DET (Mn, 34,100) at 37° C.

FIG. 2 is an illustration of what GPC traces of PEGDA258-DET (Mn, 34,100) are after in vitro hydrolysis at 37° C.; (a) original, (b) at pH 7.4 for 42 h, and (c) at pH 5.0 for 42 h.

FIG. 3 is an illustration of PEGDA575-DET (Mn, 82,700) after in vitro hydrolysis at 37° C.; (a) original, (b) at pH 7.4 for 42 h, and (c) at pH 5.0 for 42 h.

FIG. 4 ¹H-NMR spectra of PEGDA258-DET (Mn: 36,900, PDI: 1.58) with terminal diacrylates (a), dithiols (b) and the (b) after D₂O exchange (c).

FIG. 5 is an illustration of the optical transmittance of the aqueous solutions of PEGDA575-DTT at different concentrations as a function of temperature.

FIG. 6 is an illustration of the optical transmittance of 1.0 wt % aqueous solutions of PEGDA575-DTT, PEGDA700-DTT, PEGDMA550-DTT and PEGDMA750-DTT as a function of temperature.

FIG. 7 is an illustration of the optical transmittance of 1.0 wt % aqueous solutions of PEGDA700-DTT, PEGDA700-DET, PEGDA700-DPT, PEGDA700-DBT, PEGDMA750-DET and PEGDMA750-DTT as a function temperature.

FIG. 8 is an illustration of the LCSTs of DPEGs as a function of the NaCl concentration (polymer concentration, 1 wt %).

FIG. 9 is an illustration of the pH effects on the cloud points of PEGDMA750-DTT (1 wt % in DI water).

FIG. 10 is an illustration of the swelling ratio of PEGDA700/DTT/TRIAC428 hydrogels made by the copolymerization method as a function of temperature

FIG. 11 is an illustration of the swelling ratio of PEGDA700/DTT hydrogels made from different crosslink agents using the copolymerization method as a function of temperature; the molar ratio of the acrylate from PEGDA700 to that from the crosslink agent was 4.

FIG. 12 is and illustration of the swelling ratio as a function of temperature of PEGDA700-DTT hydrogels made by the end-capping method using different crosslinking agents.

### DETAILED DESCRIPTION OF PREFERRED EMBODIMENTS

### Synthesis of Linear DPEGs from Dithiols with di(meth)acrylates or di(meth)acrylamides

Poly(PEG-diacrylate-dithiothreitol)s (PEGDA-DTT), poly(PEG-diacrylate-ethanedithiol)s (PEGDA-DET), poly(PEG-diacrylate-propanedithiol)s (PEGDA-DPT), poly(PEG-diacrylate-butanedithiol)s (PEGDA-DBT), poly(PEG-dimethacrylate-dithiothreitols (PEGDMA-DTT), poly(PEG-dimethacrylate-ethanedithiol)s (PEGDMA-DET) were prepared by Michael-type polyaddition of poly(ethylene glycol) diacrylate (PEGDA), or dimethacrylate (PEGDMA) with D, L-dithiothreitol (DTT), 1,2-ethanedithiol (DET), 1,3-propanedithiol (DPT), or 1,4-butanedithiol (DBT), respectively (Schemes 1 and 2). The synthesized DPEGs are listed in Table 1. A typical procedure is as follows. DTT (0.4830 g, 3.1313 mmol) was dissolved in 3 mL of dimethyl sulfoxide (DMSO) at room temperature. PEGDA575 (1.8000 g, 3.1310 mmol) was added to the DMSO solution. Triethylamine (TEA) (0.05 mL, 0.3587 mmol) was added dropwise to the above mixture and the polymerization was continued at room temperature for 72 h. The polymer was precipitated in ether and purified by repeated precipitations. The precipitant was dried in vacuum at 70° C. overnight. The polymer PEGDA575-DTT was characterized by GPC (Table 1) and NMR.

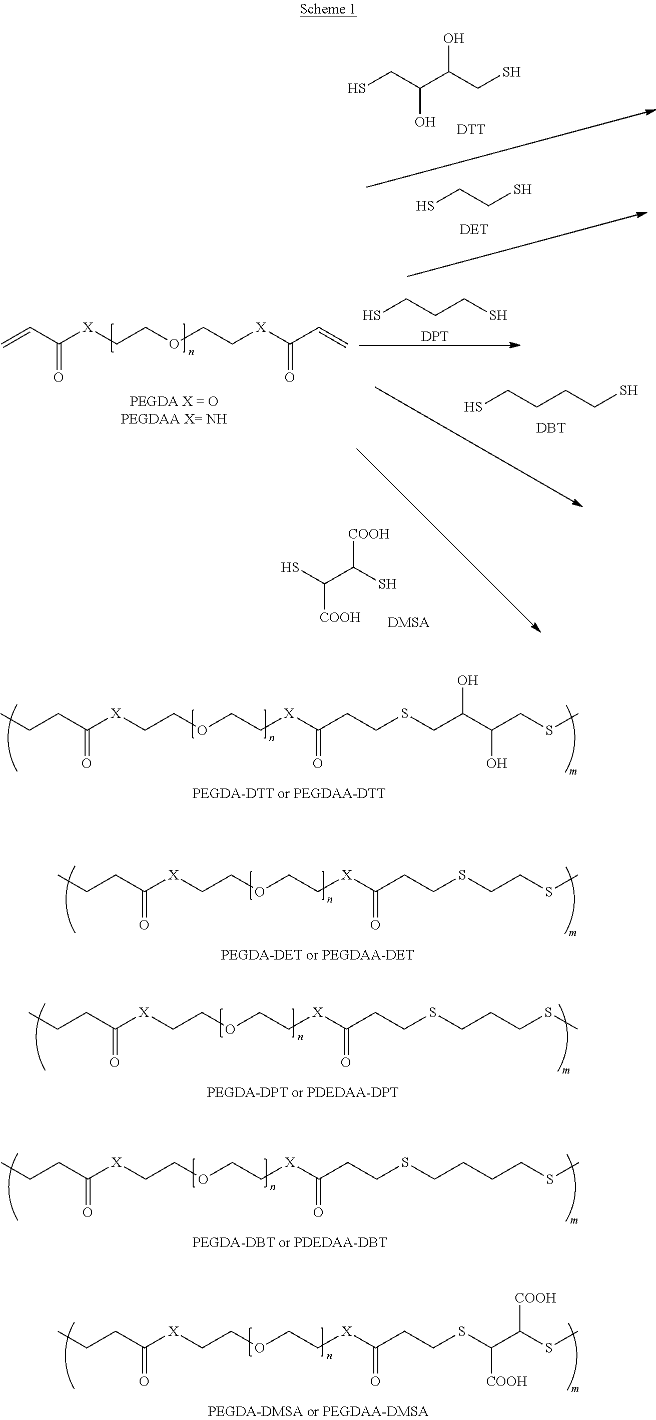

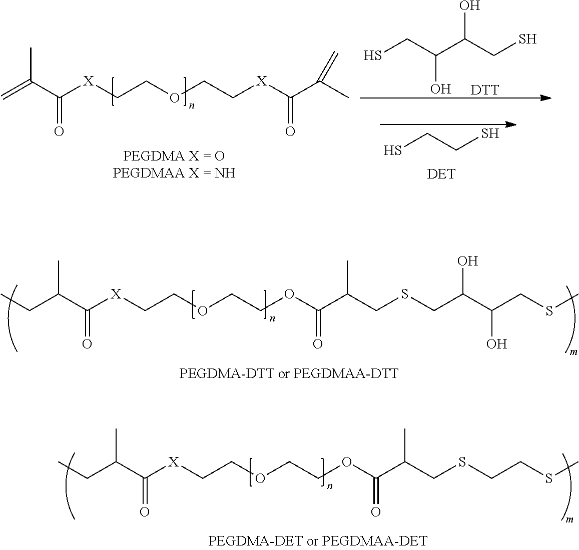

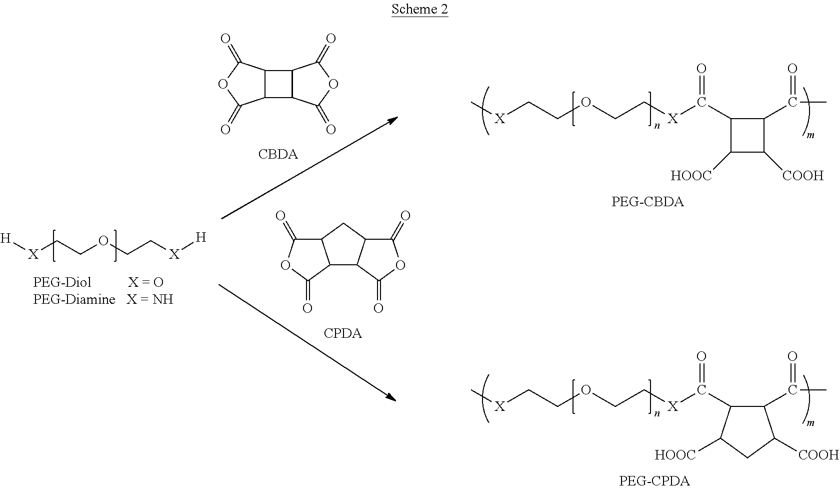

TABLE 1
The prepared DPEGs

| | PEG | | | | | |
|---|---|---|---|---|---|---|
| | macromonomer | | | | | Yield |
| Polymer Samples | | Mw | Dithiol | Mn | PDI | (%) |
| PEGDA258-DTT | PEGDA | 258 | DTT | 98,500 | 1.83 | 92 |
| PEGDA575-DTT | PEGDA | 575 | DTT | 17,400 | 1.86 | 88 |
| PEGDA700-DTT | | 700 | DTT | 10,900 | 2.01 | 82 |
| PEGDA258-DET | | 258 | DET | 34,100 | 1.56 | 94 |
| PEGDA575-DET | | 575 | DET | 34,100 | 1.56 | 94 |
| PEGDA700-DMSA | | 700 | DMSA | 30,000 | 1.8 | 90 |
| PEGDA700-DET | | 700 | DET | 20,300 | 1.79 | 85 |
| PEGDA700-DPT | | 700 | DPT | 96,100 | 1.71 | 90 |
| PEGDA700-DBT | | 700 | DBT | 10,600 | 1.65 | 86 |
| PEGDMA550-DTT | PEGDMA | 550 | DTT | 50,300 | 1.79 | 83 |
| PEGDMA750-DTT | | 750 | DTT | 15,900 | 1.86 | 79 |
| PEGDMA550-DET | | 550 | DET | 25,900 | 1.74 | 86 |
| PEGDMA750-DET | | 750 | DET | 49,000 | 1.83 | 82 |

Conditions: [PEG Macromonomer] = 1 mol/L, [dithiol] = 1 mol/L, [TEA] = 0.1 mol/L, DMSO as solvent, room temperature, 72 h.

Synthesis of Example DPEGs by the Condensation Reaction of PEG-Diol or PEG-Diamine with Dianhydride

A typical procedure is as follows: CBDA (0.4365 g, 2.2258 mmol) and PEG200 (0.4452 g, 2.2258 mmol) were dissolved in 3 mL of anhydrous DMSO at 60° C. for 72 h. The polymer was precipitated in ether and purified by repeated precipitation. The polymer was dried in vacuum oven at 70° C. overnight. Yield=92%.

Synthesis of DPEG with Terminal Dithiols (Scheme 3 b) or Diacrylates (Scheme 3 c)

An example is shown as follows: PEGDA258 (0.8207 g, 3.1810 mmol) and DET (0.2996 g, 3.1811 mmol) were dissolved in 3 mL of DMSO and stirred at room temperature. TEA (0.05 mL, 0.3587 mmol) was added dropwise to the above mixture as catalyst. After 72 h, additional DET (0.27 g, 2.87 mmol) or PEGDA258 (0.53 g, 2.05 mmol) was added to the polymerization solution and stirred at room temperature for another 30 h. The polymer was precipitated in ether for three times and dried under vacuum overnight (Mn: 36,900, PDI: 1.58). The polymers with terminal diacrylates (PEGDA258-DET-diacrylates) or dithiols (PEGDA258-DET-dithiols) were analyzed by ¹H-NMR. To confirm the terminal dithiol groups, deuterium oxide (D₂O) exchange experiment was carried out by the following procedure: D₂O (0.2 mL) was added into 0.6 mL of PEGDA258-DET-dithiols CDCl₃ solution and mixed well. After centrifuged at 2,500 rpm, PEGDA258-DET-dithiols CDCl₃ solution was collected for ¹H-NMR measurement.

Synthesis of DPEG with Terminal Dipyridyl Disulfides (Scheme 3 d)

The DPEG with terminal dipyridyl disulfide groups (PEGDA258-DET-dipyridyl disulfides) was synthesized as follows: PEGDA258-DET-dithiols (Mn: 36,900, PDI: 1.58, 2.47 g, 0.067 mmol) and 2,2-dipyridyl disulfide (0.059 g, 0.27 mmol) were dissolved in 20 mL of methanol and stirred at room temperature for 12 h. The mixture solution was precipitated in ether for three times. The precipitate was dried under a vacuum overnight.

### Conjugation of Folic Acid to DPEG (Scheme 3 e)

Introducing folic acid targeting groups to the DPEG is as follows: folic acid (2.45 g, 5.55 mmol), NHS (1.28 g, 11.12 mmol), DCC (1.37 g, 6.64 mmol) and TEA (1.86 mL, 13.34 mmol) were dissolved in 30 mL of anhydrous DMSO and stirred at room temperature overnight. The mixture was precipitated in ether for three times to isolate folate-NHS ester. Folate-NHS ester (1.21 g, 2.25 mmol) and cysteamine (0.21 g, 2.70 mmol) were dissolved in 10 mL of anhydrous DMSO. TEA (0.32 mL, 2.29 mmol) was added to the reaction mixture. The reaction was stirred for 12 h and the solution was precipitated in water for three times to obtain folate-cysteamide. PEGDA258-DET-dipyridyl disulfides (Mn: 36,900, PDI: 1.58, 2.03 g, 0.055 mmol) and folate-cysteamide (0.11 g, 0.22 mmol) were dissolved in 20 mL of DMSO and stirred at room temperature for 48 h. The polymer was repeatedly precipitated in ether until TLC (solvent system: n-propanol: water: ammonium hydroxide=8:1:2, by volume) showed no sign of free folate cysteamide. PEGDA258-DET-difolates was obtained in a yield of 81%.

### Conjugation of CPT to PEGDA-DTT

Conjugation of CPT to the DPEG with pendant hydroxyl groups is shown in Scheme 4. A typical procedure is as follows: The PEGDA700-DTT (Mn=10,900, 5.24 g, 0.48 mmol) and succinic anhydride (2.50 g, 24.98 mmol) were dissolved in 50 mL of dry DMF and stirred at 60° C. for 72 h until all hydroxyl groups were reacted. The resulting PEGDA700-DTT functionalized with pendant carboxylic acid groups (PEGDA700-DTT-acid) was isolated in ether and dried under high vacuum at 60° C. overnight.

The PEGDA700-DTT-acid reacted with CPT catalyzed by DCC/DMAP at different carboxylic acid/CPT molar ratios produced DPEG carrying different amounts of CPT molecules per chain. A typical example is as follows: the PEGDA700-DTT-acid (1.42 g, 0.11 mmol), DCC (0.35 g, 1.65 mmol), DMAP (17.1 mg, 0.14 mmol) and CPT (0.50 g, 1.43 mmol) were dissolved in 20 mL of DMSO and stirred at room temperature for 48 h. The polymer was repeatedly precipitated in ether until the thin layer chromatograph (TLC) (solvent system: chloroform: acetone=2:1, by volume) showed no sign of free CPT in the PEGDA700-DTT-CPT conjugate. Yield=86%. ¹H-NMR analysis showed that the DPEG-CPT conjugate had 12 CPT molecules per chain (PEGDA700-DTT-12CPT). The conjugate with one CPT molecule per chain (PEGDA700-DTT-1CPT) was also prepared by the same procedure by adding less CPT.

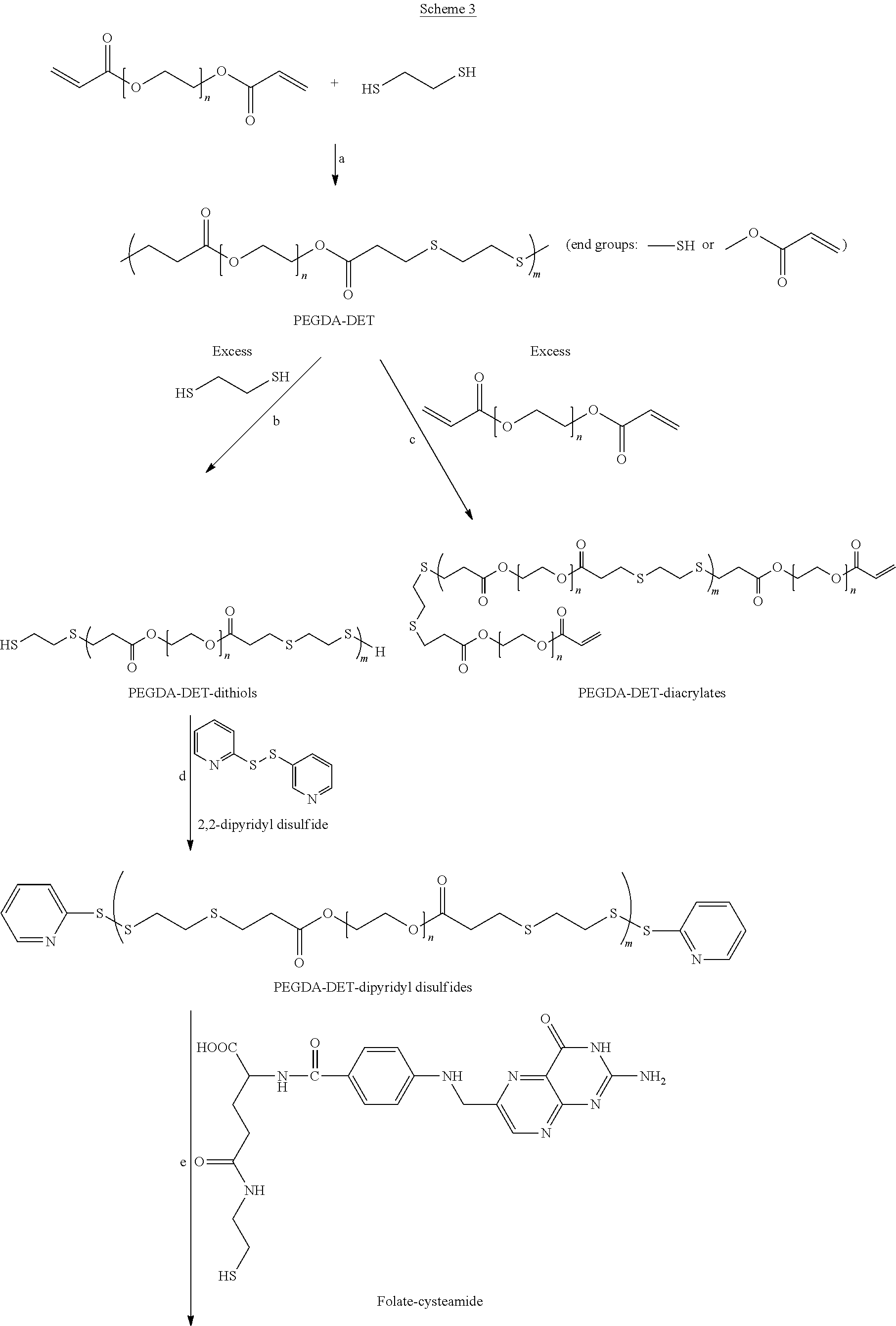

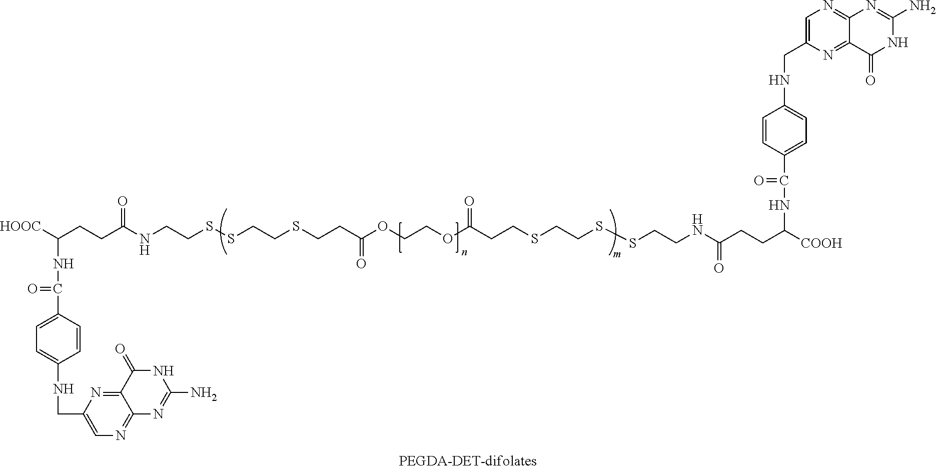

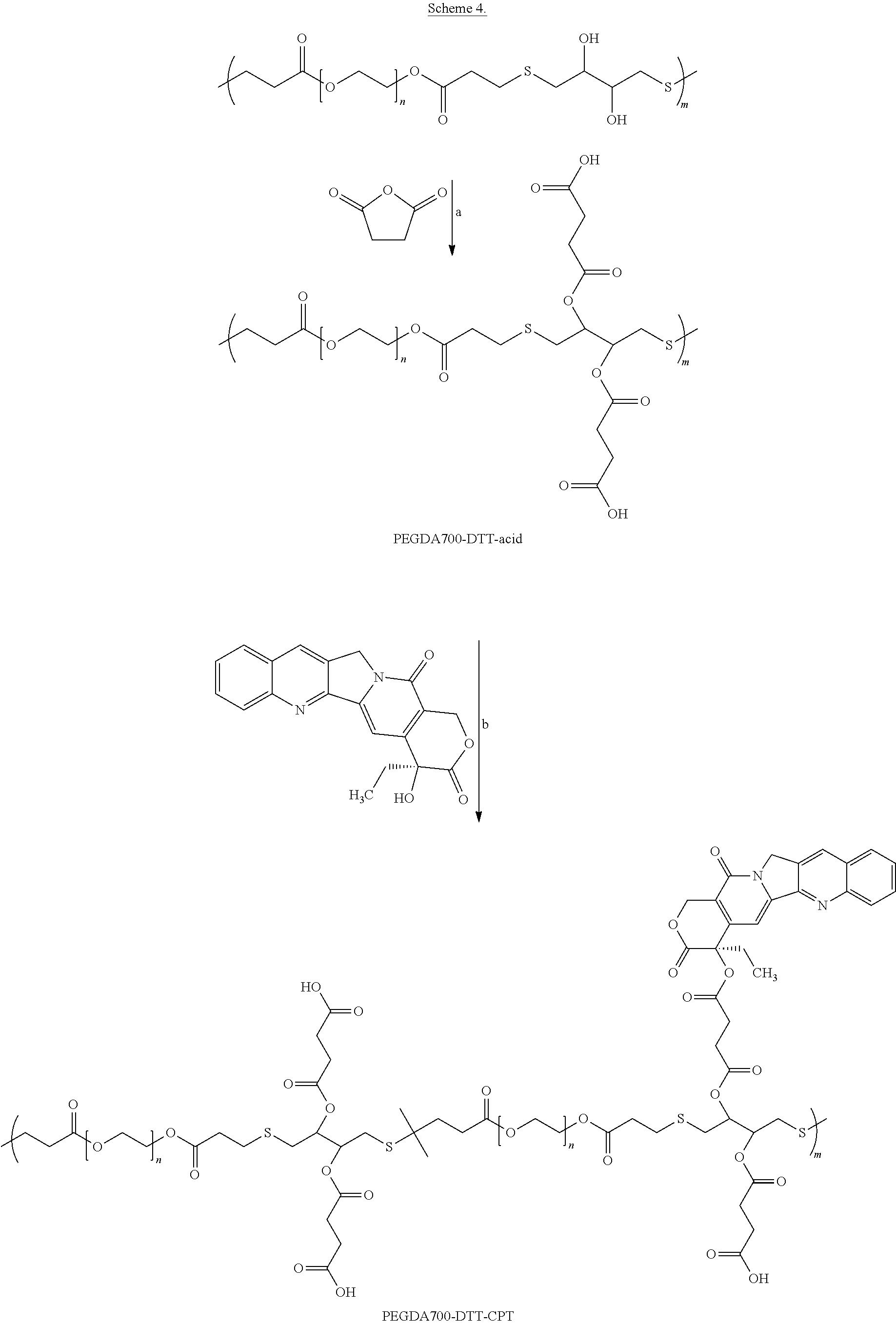

### Polymer Fractionation to Reduce Polydispersity

Generally, all tested dithiols (DTT, DET, DPT, and DBT) could react with PEGDA to produce high molecular weight DPEGs in high yields. Only DTT and DET, however, reacted with PEGDMA to produce high molecular weight DPEGs (Schemes 1 and 2). The molecular weights of the obtained DPEGs ranged from 10,000 to 90,000, depending on the dithiols and the di(meth)acrylates (Table 1). The polydispersity was about 1.7 to 2, which is typical for polymers obtained from condensation polymerization. Low polydispersed polymers could be easily obtained by repeated fractionation. For example, with CHCl₃ as solvent and ether as precipitant, 3-step fractionations of PEGDMA750-DET with Mn of 49,000 and PDI of 1.83 produced fractions with lower PDIs (Table 2).

TABLE 2
3-Step fractionation results of PEGDMA750-DET

| | Mn | PDI | Yield (%) |
|---|---|---|---|
| Original | 49,000 | 1.83 | |
| 1^{st}-fraction | 56,700 | 1.63 | 25 |
| 2^{nd}-fraction | 20,600 | 1.25 | 35 |
| 3^{rd}-3 fraction | 13,500 | 1.18 | 17 |

### Examples Demonstrating DPEG Hydrolysis

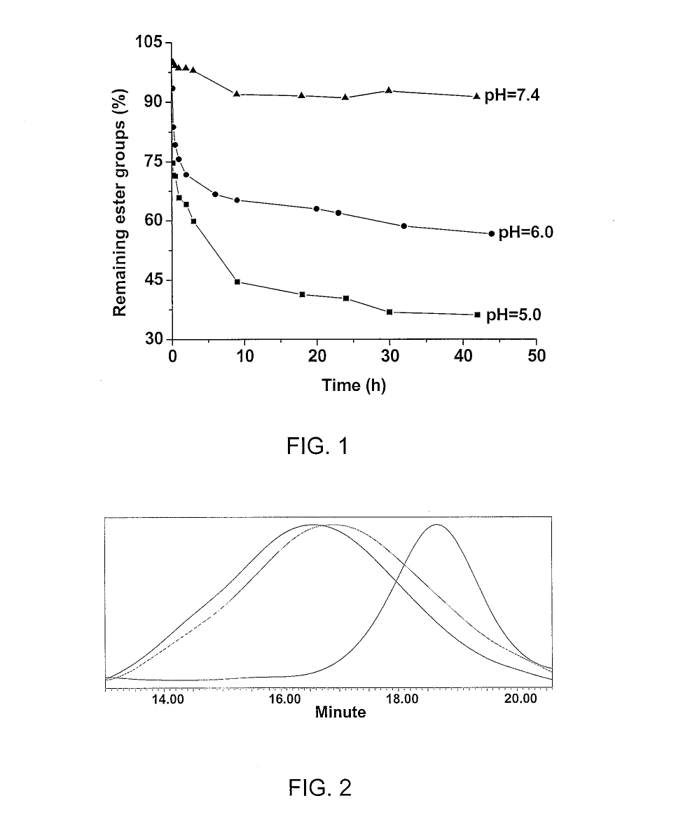

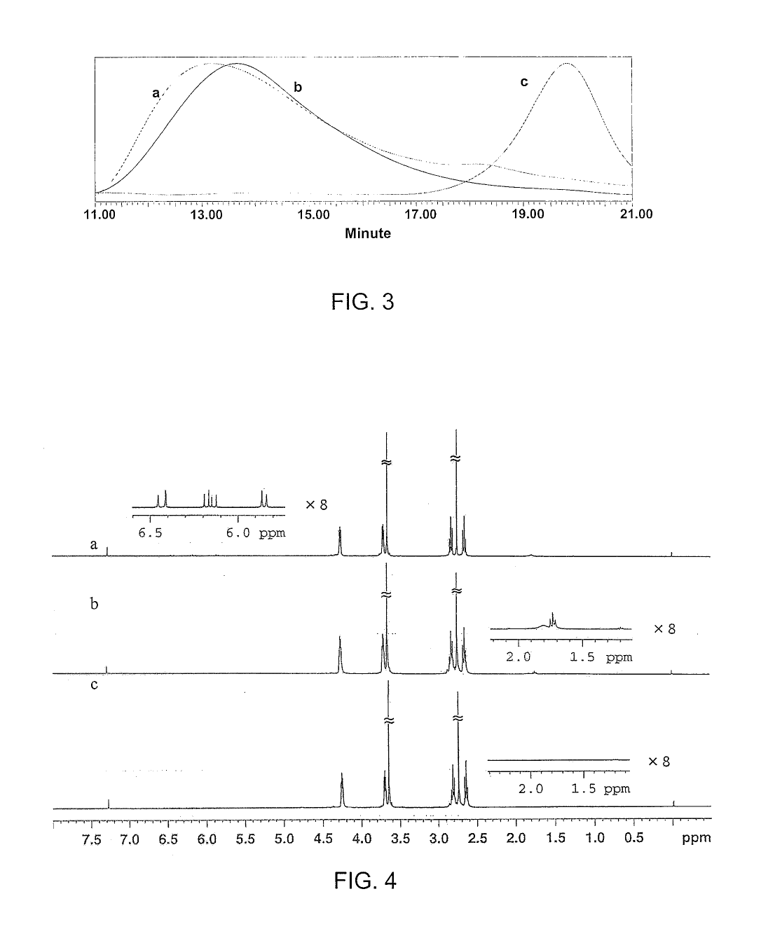

DPEG's are degradable because they are hydrolysable. Specifically, DPEG backbone contains β-thioester (—SCH₂CH₂COO— or —SCH₂CH(CH₃)COO—) groups that promote hydrolysis. The hydrolysis of DPEGs was tested at pH 7.4, 6.0 and 5.0 and monitored by measuring the decrease of the ester bonds using ¹H-NMR. FIG. 1 shows the hydrolysis of PEGDA258-DET at different pHs. At pH 7.4, PEGDA258-DET was relatively stable. Only 8.7% of the ester groups hydrolyzed after 42 h. While at pH 5.0, PEGDA258-DET hydrolyzed quickly. About 25.3% in 15 min and 55.48% in 9 h of the ester groups were hydrolyzed. At pH 6.0, PEGDA258-DET hydrolyzed faster than at pH 7.4, but more slowly than at pH 5.0. About 34.8% of the ester groups hydrolyzed in 9 h. Correspondingly, the Mn of PEGDA258-DET only decreased slightly at pH7.4 even after 42 h, but it hydrolyzed to oligomers (Mn, ˜2000) after 42 h at pH5 (FIG. 2). Similar results were also obtained from PEGDA575-DET (Mn, 82,700) (FIG. 3). These results indicate that the DPEGs are relatively stable at pH 7.4 while degrade rapidly at pH 5.0 and pH 6. Thus, high molecular weight DPEGs can be used as drug carriers for effective cancer targeting while they can degrade into oligomers in lysosomes for effective renal clearance.

### Examples Demonstrating DPEG Functionalization

In addition to the functional groups introduced by using dithiols having functional groups, such as DTT, the DPEGs could easily be functionalized with terminal (meth)acrylate or thiol groups (Scheme 3). The ratio of di(meth)acrylate monomer to dithiol monomer was first kept at 1/1 molar ratio to make a high molecular weight polymer. After a desirable molecular weight was reached (e.g., PEGDA258-DET, Mn: 36,900, PDI: 1.58), an excess of dithiol or di(meth)acrylate monomer was added to the reaction solution to cap the polymer ends with either thiol or (meth)acrylate. Typical ¹H-NMR spectra are shown in FIG. 4. The peaks at about 5.8 ppm, 6.1-6.2 ppm, and 6.4 ppm were present in the NMR spectrum of PEGDA258-DET-diacrylates, indicating the existence of terminal acrylate groups. The molecular weight calculated from the integrations of the acrylate peaks and the ester peak was about half of the values measured by GPC (Mn: 36,900, PDI: 1.58), suggesting that the polymer chains indeed have acrylates at the both ends. ¹H-NMR spectrum of PEGDA258-DET-dithiols had a peak at 1.7 ppm, which disappeared after D₂O exchange, indicating the existence of terminal dithiol groups. The terminal thiol groups were further continued by the reaction with pyridyl disulfide (Scheme 3). The presence of 2-pyridyldithio group in the polymer again confirmed the presence of the terminal thiol groups. The calculation from the integrations also showed that there were pyridyl disulfide groups at the both ends.

The use of terminal thiol groups for conjugation was demonstrated by introducing folic acid targeting groups (Scheme 3 d and e). The PEGDA258-DET with terminal 2-pyridyldisulfides reacted with folic acid-cysteamide prepared from folic acid and cysteamine and formed the disulfide bonds, anchoring the folic acid moieties to the DPEG (PEGDA258-DET-difolates). The presence of folic acid moieties was confirmed by NMR (experimental section) and was about 2 folic acid groups per chain.

### Synthesis of DPEG Hydrogels

The DPEG hydrogels were synthesized by an in-situ copolymerization method or an end-capping method using crosslinking agents of pentaerythritol tetraacrylate (TEAC) or trimethylolpropane ethoxylate triacrylate (TRIAC) with molecular weight of 428 (TRIAC428), 604 (TRIAC604), 912 (TRIAC912) (Scheme 5).

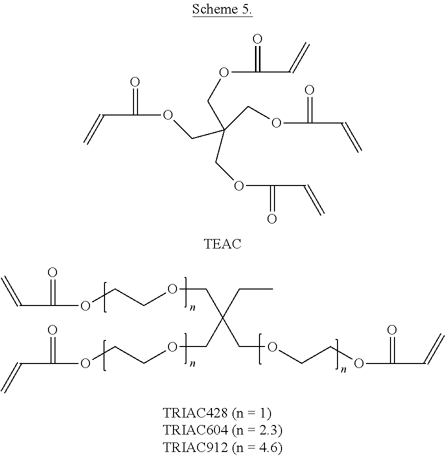

In the in-situ copolymerization method, PEGDA700, DTT and a crosslinking agent were copolymerized to form gels. TRIAC428 at 5, 10 or 15 wt-% of PEGDA700 were used. The corresponding molar ratios of the acrylate from PEGDA700 to that from TRIAC428 were 8, 4, and 3 respectively. The amounts of other crosslinking agents in the copolymerization were calculated according to the molar ratios. A typical procedure is as follows. PEGDA700 (2.1479 g, 3.0685 mmol), TRIAC912 (0.2289 g, 0.2510 mmol) and DTT (0.5314 g, 3.445 mmol) were dissolved in 3 mL of DMSO at room temperature. TEA (0.05 mL, 0.3587 mmol) was added dropwise to the above mixture and the crosslinking polymerization was continued at room temperature for 72 h. The solids were extracted for 24 h with 250 ml of acetone using a Soxhlet extractor. The insoluble solid in a yield of 92% was dried in vacuum at 70° C. overnight.

In the end-capping method, the DPEG with terminal thiol groups at the both ends (DPEG-dithiols) was first synthesized as the precursor. A crosslinking agent and TEA were then added to form the gels (Scheme 6). The molar ratio of the acrylate in the crosslinking agent to the thiol group in the DPEG-dithiols was kept close to 1/1. A typical procedure is as follows. DET (0.3353 g, 3.5606 mmol) and PEGDA700 (2.4924 g, 3.5606 mmol) were dissolved in 3 mL of DMSO and stirred at room temperature. TEA (0.05 mL, 0.3587 mmol) was added dropwise to the mixture as catalyst. After 72 h, additional DET (0.31 g, 3.29 mmol) was added to the polymerization solution and stirred at room temperature for another 30 h. The polymer was precipitated in ether for three times and dried under vacuum overnight. PEGDA700-DET with terminal thiol groups at both ends (PEGDA700-DET-dithiols, Mn: 22,900, PDI: 1.62) was obtained. PEGDA700-DET-dithiols (1.6450 g, 0.0718 mmol) and TEAC (12.6490 mg, 0.0359 mmol) were dissolved in 10 mL of DMSO at room temperature. TEA (2.84, 0.02 mmol) was added dropwise to the above mixture and the polymerization was continued at 35° C. for 96 h. The resulting soft solid was extracted for 24 h with 250 ml of acetone using a Soxhlet extractor. The insoluble solid in a yield of 83% was dried in vacuum at 70° C. overnight.

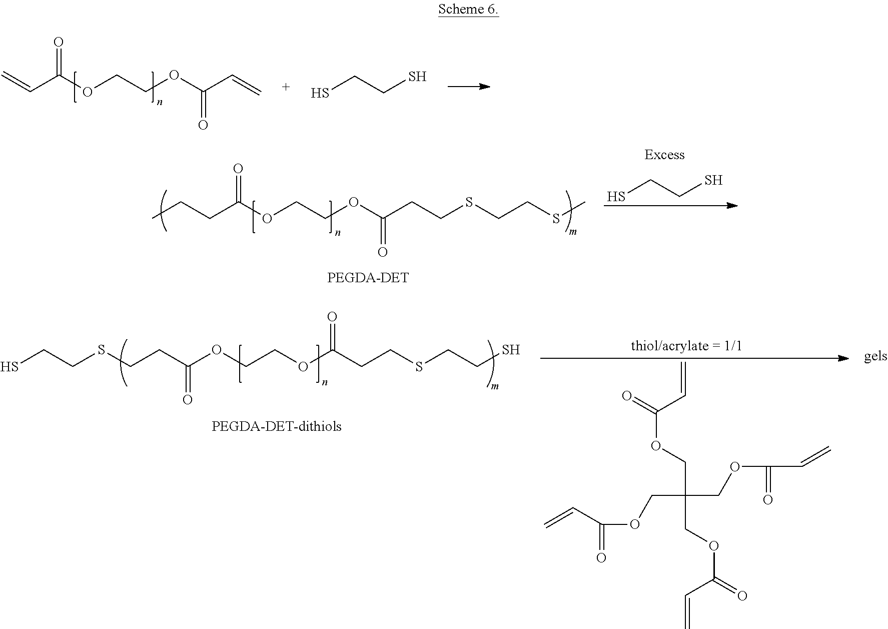

The DPEG gels resulted from the both methods are summarized in Tables 3 and 4.

TABLE 3
The DPEG gels synthesized by the in-situ copolymerization method

| | | | Molar ratio | |
|---|---|---|---|---|
| | | | of acrylate | |
| | | | from | |
| | | | PEGDA | |
| | | | to that | |
| | | | from the | |
| Crosslinking agent | | | crosslinking | |
| Type | wt % | Hydrogels | agent | Yield |
| TRIAC428 | 5 | PEGDA700/DTT/ | 8 | 91% |
| | | TRIAC428-5% | | |
| | 10 | PEGDA700/DTT/ | 4 | 87% |
| | | TRIAC428-10% | | |
| | 17 | PEGDA700/DTT/ | 3 | 92% |
| | | TRIAC428-15% | | |
| TRIAC604 | 7 | PEGDA700/DTT/ | 8 | 85% |
| | | TRIAC604-7% | | |
| | 14 | PEGDA700/DTT/ | 4 | 89% |
| | | TRIAC604-14% | | |
| | 21 | PEGDA700/DTT/ | 3 | 94% |
| | | TRIAC604-21% | | |
| TRIAC912 | 10 | PEGDA700/DTT/ | 8 | 92% |
| | | TRIAC912-10% | | |
| | 21 | PEGDA700/DTT/ | 4 | 93% |
| | | TRIAC912-21% | | |
| | 32 | PEGDA700/DTT/ | 3 | 91% |
| | | TRIAC912-32% | | |
| TEAC | 3 | PEGDA700/DTT/TEAC-3% | 8 | 86% |
| | 8 | PEGDA700/DTT/TEAC-8% | 4 | 94% |
| | 12 | PEGDA700/DTT/TEAC-12% | 3 | 93% |

Conditions: Acrylate/thiol (in molar) = 1. [dithiol] = 1 mol/L, [TEA] = 0.1 mol/L, DMSO as solvent, room temperature, 72 h.

TABLE 4
The DPEG hydrogels synthesized by the end-capping method using
PEGDA700-DET-dithiols (Mn: 22,900, PDI: 1.62)

| DPEG Hydrogels | Crosslinking Agent | Yield (%) |
|---|---|---|
| PEGDA700-DET/TRIAC428 | TRIAC428 | 75% |
| PEGDA700-DET/TRIAC604 | TRIAC604 | 52% |
| PEGDA700-DET/TRIAC912 | TRIAC912 | 67% |
| PEGDA700-DET/TEAC | TEAC | 83% |

Conditions: Molar ratio of acrylate in the crosslinking agent to the thiol groups in PEGDA700-DET-dithiols was 1/1. [PEGDA700-DET-dithiols] = 0.04 mol/L, [TEA] = 0.01 mol/L, DMSO as solvent, 35° C., 96 h.

Ionic strength and pH effects on the phase transition temperature were tested by measuring the cloud points of the DPEG (1 wt %) in NaCl solutions at 0.15, 0.5, 1.0, and 2.0 mol/L or at pH 6.0, 7.0, and 8.0.

The swelling ratio (%) of the hydrogels was measured in terms of the percent of absorbed water by the dry gels. The gel particles were equilibrated in water for 24 h. The hydrated particles were carefully taken out from the solution, wiped with a filter paper to remove the free water on the surface and then weighted. Swelling ratio (%) of a sample was calculated by:

$$Swelling ratio ( \% ) = \frac{w - {w}_{0}}{{w}_{0}}$$

where w is the hydrogel weight at equilibrium and w₀ is the weight of the dry gel.

### Examples of Results

All tested dithiols (DTT, DET, DPT, and DBT) could react with PEGDA to produce high molecular weight DPEGs in high yields. Only DTT and DET, however, reacted with PEGDMA to produce high molecular weight DPEGs (Schemes 1 and 2). The molecular weights of the obtained DPEGs ranged from 10,000 to 90,000, depending on the dithiols and the di(meth)acrylates (Table 1). The polydispersity was about 1.7 to 2, which is typical for polymers obtained from condensation polymerization. Low polydispersed polymers could be easily obtained by repeated fractionation. For example, with CHCl₃ as solvent and ether as precipitant, 3-step fractionations of PEGDMA750-DET with Mn of 49,000 and PDI of 1.83 produced fractions with lower PDIs (Table 2). Their degradation was confirmed by the hydrolysis experiments. DPEG is stable at the neutral pH but hydrolyzes quickly at acidic pHs.

DPEGs can be made to carry multifunctional groups. In addition to the functional groups introduced by using dithiols having functional groups, such as hydroxyl groups (DTT) and carboxylic acid groups (DMSA), the DPEGs could easily be functionalized with terminal (meth)acrylate or thiol groups (Scheme 3). The ratio of di(meth)acrylate monomer to dithiol monomer was first kept at 1/1 molar ratio to make a high molecular weight polymer. After a desirable molecular weight was reached (e.g., PEGDA258-DET, Mn: 36,900, PDI: 1.58), an excess of dithiol or di(meth)acrylate monomer was added to the reaction solution to cap the polymer ends with either thiol or (meth)acrylate. Typical ¹H-NMR spectra are shown in FIG. 4. The peaks at about 5.8 ppm, 6.1-6.2 ppm, and 6.4 ppm were present in the NMR spectrum of PEGDA258-DET-diacrylates, indicating the existence of terminal acrylate groups. The molecular weight calculated from the integrations of the acrylate peaks and the ester peak was about half of the values measured by GPC (Mn: 36,900, PDI: 1.58), suggesting that the polymer chains indeed have acrylates at the both ends. ¹H-NMR spectrum of PEGDA258-DET-dithiols had a peak at 1.7 ppm, which disappeared after D₂O exchange, indicating the existence of terminal dithiol groups. The terminal thiol groups were further confirmed by the reaction with pyridyl disulfide (Scheme 3). The presence of 2-pyridyldithio group in the polymer again confirmed the presence of the terminal thiol groups. The calculation from the integrations also showed that there were pyridyl disulfide groups at the both ends.

The use of terminal thiol groups for conjugation was demonstrated by introducing folic acid targeting groups (Scheme 3 d and e). The PEGDA258-DET with terminal 2-pyridyldisulfides reacted with folic acid-cysteamide prepared from folic acid and cysteamine and formed the disulfide bonds, anchoring the folic acid moieties to the DPEG (PEGDA258-DET-difolates). The presence of folic acid moieties was confirmed by NMR (experimental section) and was about 2 folic acid groups per chain.

The use of the hydroxyl groups in DTT-based DPEG was demonstrated by conjugation of CPT (Scheme 4). PEGDA700-DTT was first reacted with succinic anhydride to convert the hydroxyl groups into acid groups (PEGDA700-DTT). It is important to ensure all the hydroxyl groups reacted; otherwise, crosslinking would occur in the next step of CPT conjugation. CPT was anchored to the chains using conventional DCC/DMAP catalyzed reaction. The amount of CPT per chain was controlled by CPT/carboxylic acid ratio. The resulting PEGDA700-DTT-CPT was analyzed by NMR (Experimental section). PEGDA700-DTT-CPT samples having 12 CPT/chain and 1 CPT/chain were prepared.

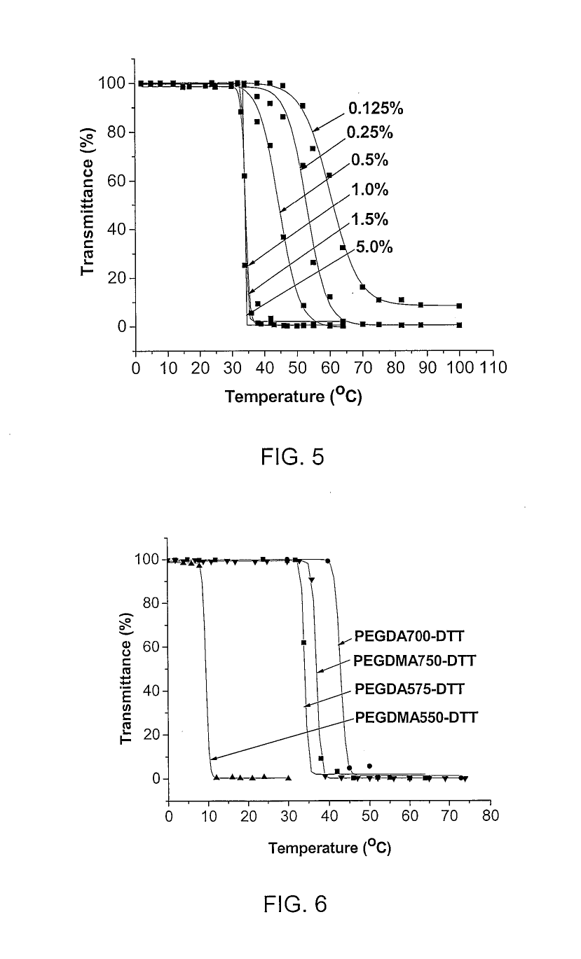

These DPEGs were found to have thermosensitive properties. A clear solution of the DPEG became cloudy upon heating. A typical optical transmittance of the solution of PEGDA575-DTT as a function of temperature is shown in FIG. 5. Clearly, the PEGDA575-DTT precipitated out from the solution at temperatures around 40-60° C., depending on its concentration. Below 1 wt %, the cloud point decreased as the polymer concentration increased. At 0.125 wt %, the cloud point was 55° C. The phase transition region was also broad. When the concentration was 1.0 wt % or higher, the cloud point of the DPEG was about 36° C. independent of the DPEG concentration. The phase transition was also sharp, within a 2° C. range. Thus, PEGDA575-DTT had a LCST at about 36° C.

The influences of molecular weight and structure of the PEG-di(meth)acrylate macromonomers on the DPEG's LCST are shown in FIG. 6. With the same dithiol (DTT), the DPEG obtained from a PEGDA with molecular weight of 700 (PEGDA700-DTT) had a LCST of 45° C., 9° C. higher than that of DPEG obtained from PEGDA575 (PEGDA575-DTT). Similarly, the DPEG from PEGDMA with Mn of 750 (PEGDMA750-DTT) had a LCST of 39° C., significantly higher than that of the DPEG from PEGDMA550 (PEGDMA550-DTT). Thus, the LCST of the DPEG is very sensitive to the number of the ethylene glycol units in the PEG-(meth)acrylate macromonomers (Table 1). A longer PEG chain in the PEG-di(meth)acrylate resulted in a higher LCST of the polymer. FIG. 6 also shows that the DPEG made from PEGDMA had a lower LCST than that made from PEGDA with the same PEG molecular weight (or n in Table 1) and dithiol. The cloud point of PEGDA700-DTT was 6° C. higher than that of PEGDMA750-DTT, and the cloud point of PEGDA575-DTT was 22° C. higher than that of PEGDMA550-DTT.

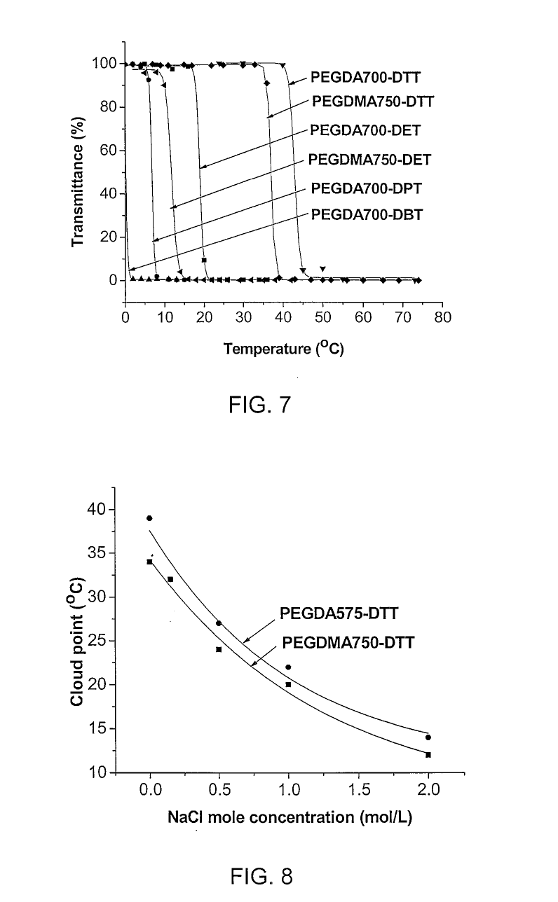

The phase transitions of DPEGs obtained from PEGDA700 with different dithiols are shown in FIG. 7. A long methylene-chain in the dithiol decreased the LCST of the resulting DPEG. The LCSTs of PEGDA700-DET, PEGDA700-DPT and PEGDA700-DBT were 20, 8 and 2° C., respectively. Introducing hydroxyl groups to the dithiol increased the LCST of the DPEG. PEGDMA750-DTT had a LCST of 39° C., but the LCST of PEGDMA750-DET was 14° C., a 25° C. difference. A Similar difference was found in PEGDA700-DTT and PEGDA700-DET.

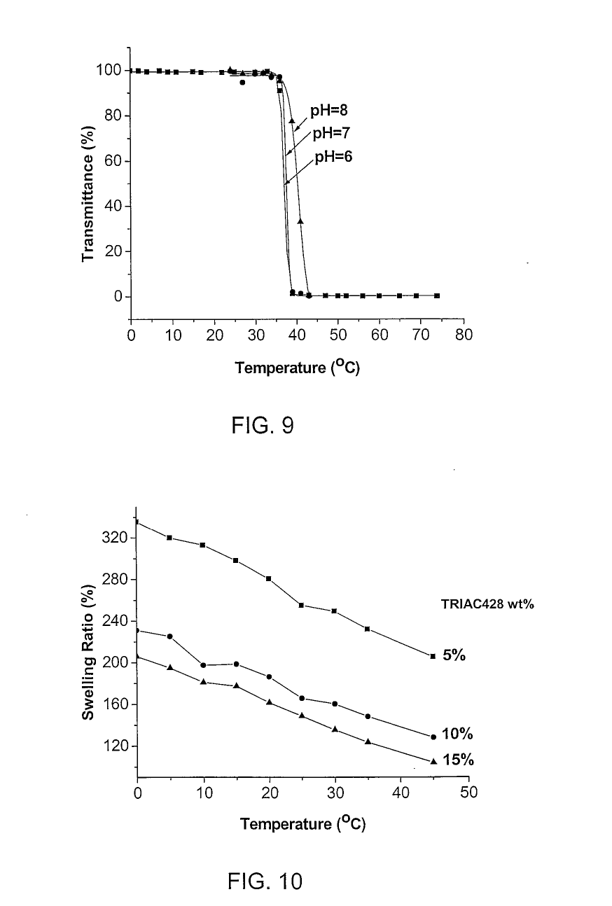

The effects of ionic strength on the LCSTs of DPEGs were tested by measuring the LCSTs in the presence of varied concentrations of NaCl (FIG. 8). The presence of NaCl significantly decreased the LCSTs of DPEGs. This is consistent with the “salt out” effect found in other thermoresponsive polymers.³² FIG. 9 shows that the solution pH had little effect on the LCST of PEGDMA750-DTT.

### Thermoresponsive DPEG Hydrogels

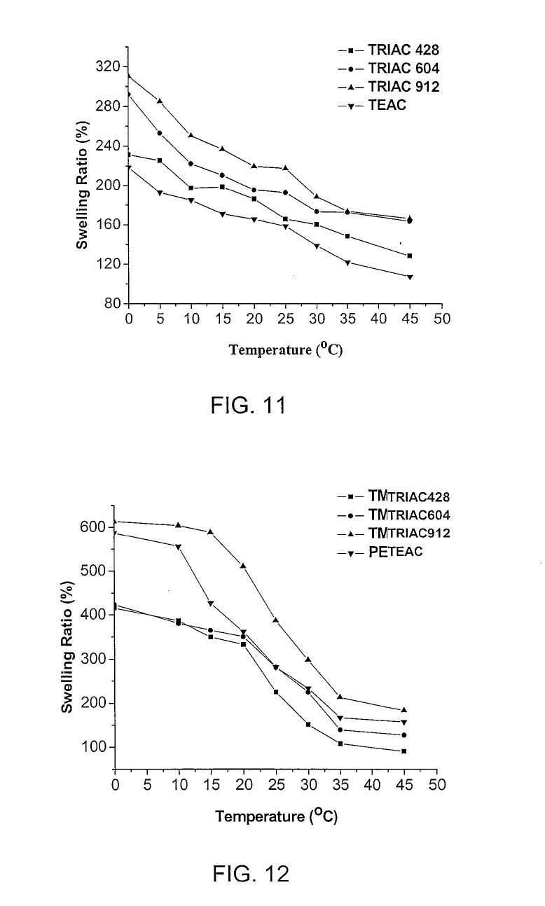

DPEG gels were first prepared by in-situ polymerization of PEGDA700 and DTT in the presence of a crosslinking agent TRIAC or TEAC. The copolymerization with 5% or more crosslinking agent produced gels in high yields (Table 1). The gels swelled at low temperatures but deswelled at elevated temperatures. The thermoresponsive property in terms of the swelling ratio as a function temperature of the hydrogels is shown in FIGS. 10 and 11. In contrast to the sharp phase transition at 45° C. of noncrosslinked PDEDA700-DTT (FIG. 6), the PEGDA700/DTT/TRIAC428 hydrogels deswelled gradually as the temperature increased. The deswelling started even at around 0° C., much lower than the LCST of PEGDA700-DTT. This is because that TRIAC428 contains only one ethylene glycol unit on average in each of its branch (n=1 in Scheme 5) and thus it is a relatively hydrophobic molecule. The incorporation of TRIAC428 into PEGDA700-DTT chains lowers the LCST. TRIAC428 units were randomly distributed in the PEGDA700-DTT chains, causing the transition temperature to span from 0 to 45° C.

As expected, the swelling ratio of the hydrogels decreased as the TRIAC428 content in the gel increased from 5% to 15% (FIG. 10). At 5° C., the swelling ratio of the hydrogels was 320%, 225% and 194%, respectively when TRIAC428 was 5 wt %, 10 wt %, and 15 wt % of PEGDA700. The swellability of the hydrogels was also affected by the type of crosslinking agents (FIG. 7). As the TRIAC molecular weight, i.e., the ethylene glycol unit n shown in Scheme 5, increased, the swelling ratio of the resulting hydrogel increased. For example, at the molar ratio of the acrylate from PEGDA700 to that from the crosslink agent of 4, the swelling ratios at 0° C. of the hydrogels with TRIAC 912, TRIAC 604, TRIAC 428 and TEAC were 310%, 292%, 231% and 218%, respectively. The hydrogel from TEAC had even lower swelling ratios at the same temperature.

The cocondensation polymerization in the presence of crosslinking agents produced thermoresponsive hydrogels, but their phase transitions were broad due to the random distribution of the crosslinking agents in the DPEG chains. Thus, an end-capping method was used to synthesize the gels. In this method, the DPEG chains with terminal thiol groups at the both ends were first synthesized and then reacted with a crosslinker agent TRIAC or TEAC (Scheme 6). The DPEG chains were thus minimally disturbed to retain their thermoresponsive properties. FIG. 12 shows the swelling ratio as a function of temperature of hydrogels made from PEGDA700-DET with different crosslinking agents (Table 2). Noncrosslinked linear PEGDA700-DET had a LCST of 20° C. (FIG. 7).

The hydrogels made by the end-capping method had much higher swelling ratios than those made by the copolymerization method (FIG. 12). For example, PEGDA-DET-TRIAC912 and PEGDA-DET-TEAC had a swelling ratio about 600% at 10° C. In contrast to the gradual deswelling of the hydrogels made by the copolymerization method, the hydrogels made by the end-capping method deswelled in a certain range of temperature close to the LCST of PEGDA700-DET even though the transition temperature region was broad compared to that of linear PEGDA700-DET. Thus, the end-capping crosslinking produced hydrogels with improved thermoresponsive properties.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1. Duncan, R. Nat. Rev. 2003, 2, 347-360.
2. Satchi-Fainaro, R.; Duncan, R.; Barnes, C. M. Adv. Polym. Sci. 2006, 193, 1-65.
3. Thatte, S.; Datar, K.; Ottenbrite, R. M. J. Bioact. Compat. Polym. 2005, 20, 585-601.
4. Qiu, L. Y.; Bae, Y. H. Pharm. Res. 2006, 23, 1-30.
5. Duncan, R. Nature Rev. Cancer 2006, 6, 688-701.
6. Khandare, J. J.; Jayant, S.; Singh, A.; Chandna, P.; Wang, Y.; Vorsa, N.; Minko, T. Bioconjugate Chem. 2006, 17, 1464-1472.
7. Seymour, L. W. Crit. Rev. Ther. Drug Carrier Sys. 1992, 9, 135-187.
8. Lukyanov, A. N.; Gao, Z.; Mazzola, L.; Torchilin, V. P. Pharm. Res. 2002, 19, 1424-1429.
9. Haag, R.; Kratz, F. Angew. Chem. Int. Ed 2006, 45, 1198-1215.
10. Rowinsky, E. K.; Rizzo, J.; Ochoa, L.; Takimota, C. H.; Forouzesh, B.; Schwartz, G.; Hammond, L. A.; Patnaik, A.; Kwiatek, J.; Goetz, A.; Denis, L.; McGuire, J.; Tolcher, A. W. J. Clin. Oncol. 2003, 21, 148-157.
11. Hamann, P. R.; Hinman, L. M.; Beyer, C. F.; Greenberger, L. M.; Lin, C.; Lindh, D.;
Menendez, A. T.; Wallace, R.; Durr, F. E.; Upeslacis, J. Bioconjugate Chem. 2005, 16, 346-353.
12. Duncan, R.; Vicent, M. J.; Greco, F.; Nicholson, R. I. Endocr.-Relat. Cancer 2005, 12, S189-99.
13. Nan, A.; Ghandehari, H.; Hebert, C.; Siavash, H.; Nikitakis, N.; Reynolds, M.; Sauk, J. J. J. Drug Targeting 2005, 13, 189-197.
14. Non, A.; Kopecek, J. Adv. Drug Deliv. Rev. 2005, 57, 609-636.
15. Yu, D.; Peng, P.; Dharap, S. S.; Wang, Y.; Mehlig, M.; Chandna, P.; Zhao, H.; Filpula, D.; Yang, K.; Borowski, V.; Borchard, G.; Zhang, Z.; Minko, T. J. Control. Release 2005, 110, 90-102.
16. Dharap, S. S.; Qiu, B.; Williams, G. C.; Sinko, P.; Stein, S.; Minko, T. J. Control. Release 2003, 91, 61-73.
17. Rodrigues, P. C. A.; Beyer, U.; Schumacher, P.; Roth, T.; Fiebig, H. H.; Unger, C.;
Messori, L.; Orioli, P.; Paper, D. H.; Mulhaupt, R.; Kratz, F. Bioorgan. Med. Chem. 1999, 7, 2517-2524.
18. Pechar, M.; Ulbrich, K.; Jelinkova, M.; Rihova, B. Mcicromol. Biosci. 2003, 3, 364-372.
19. Veronese, F. M.; Schiavon, O.; Pasut, G.; Mendichi, R.; Andersson, L.; Tsirk, A.; Ford, J.; Wu, G.; Kneller, S.; Davies, J.; Duncan, R. Bioconjugate Chem. 2005, 16, 775-784.
20. Schoemnakers, R. G.; van de Wetering, P.; Elbert, D. L.; Hubbell, J. A. J. Control. Release 2004, 95, 291-300.
21. Riebeseel, K.; Biedermann, E.; Loeser, R.; Breiter, N.; Hanselmann, R.; Muelhaupt, R.; Unger, C.; Kratz, F. Bioconjugate Chem. 2002, 13, 773-785.
22. Yamaoka, T.; Tabata, Y.; Wada, Y. J. Pharm. Sci. 1994, 83, 601-606.
23. Schneider, P.; Korolenko, T.; Busch, U. Microsc. Res. Techn. 1997, 36, 253-275.
24. Helmlinger, G.; Yuan, F.; Dellian, M.; Jain, R. K. Nat. Med. 1997, 3, 177-182.
25. Jain, R. K. J. Control. Release 2001, 74, 7-25.
26. Reijngoud, D. J.; Tager, J. M. Biochim. Biophys. Acta 1977, 472, 419-449.
27. Lutz, J.-F.; Hoth, A. Macromolecules 2006, 39, 893-896.
28. Lutz, J.-F.; Akdemir, O.; Hoth, A. J. Am. Chem. Soc. 2006, 128, 13046-13047.
29. Hua, F.; Jiang, X.; Li, D.; Zhao, B. J. Polym. Sci. Part A: Polym. Chem. 2006, 44, 2454-2467.
30. Li, D.; Jones, G. L.; Dunlap, J. R.; Hua, F.; Zhao, B. Langmuir 2006, 22, 3344-3351.
31. Zhao, B.; Li, D.; Hua, F.; Green, D. R. Macromolecules 2005, 38, 9509-9517.
32. Mao, H.; Li, C.; Zhang, Y.; Furyk, S.; Cremer, P. S.; Bergbreiter, D. E. Macromolecules 2004, 37, 1031-1036.

We claim:

1. A method for making poly(ethylene glycol) (PEG) analogues, comprising reacting PEG-di(meth)acrylates or di(meth)acrylamides with dithiols.

2. A method for making poly(ethylene glycol) (PEG) analogues, comprising reacting PEG-diols or PEG-diamines with dianhydrides.

3. A PEG analogues produced by practicing the method of claim 1 or claim 2.

4. A method of drug delivery comprising administration to a subject of a compound produced under claim 1 or claim 2 linked to a drug.

5. Compounds comprising a compound produced under claim 1 or claim 2 covalently bonded to a material selected from the group consisting of a biomolecule and a colloidal particle.

6. Thermoresponsive polymers, comprising crosslinked compounds of either claim 1 or claim 2.

7. A method as defined in claim 1, wherein the reaction is by Michael Addition.
